(19) United States
(12) Patent Application Publication
(10) Pub. No.: US 2023/0018245 A1
(43) Pub. Date: Jan. 19, 2023

(54) Skin Whitening Composition Containing Dihydro-5-Methylfuran-2(3h)-One Derivative

(71) Applicant: Epi Biotech Co., Ltd., Incheon (KR)

(72) Inventors: Jong-Hyuk Sung, Gyeonggi-do (KR); Hyunju KIM, Incheon (KR); Cheong-Yong Yun, Incheon (KR)

(73) Assignee: Epi Biotech Co., Ltd., Incheon (KR)

(21) Appl. No.: 17/633,317

(22) PCT Filed: Aug. 7, 2020
(86) PCT No.: PCT/KR2020/010491

§ 371 Date: Aug. 31, 2022

(30) Foreign Application Priority Data
Aug. 8, 2019 (KR) 10-2019-0096527
Apr. 29, 2020 (KR) 10-2020-0052217

(51) Int. Cl. A61K 8/49; A61Q 19/02; A61K 8/34; A23L 33/10

(52) U.S. Cl. CPC A61K 8/4973; A61Q 19/02; A61K 8/347; A23L 33/10; A61K 2800/92; A23V 2002/00

(57) ABSTRACT

The present invention relates to a composition for skin whitening comprising a dihydro-5-methylfuran-2(3H)-one derivative, an isomer thereof, or a pharmaceutically acceptable salt thereof having melanogenesis inhibitory activity and melanosome degradation activity. Specifically, the composition of the present invention inhibits melanogenesis and promotes melanosome degradation by means of the action of a dihydro-5-methylfuran-2(3H)-one derivative in a signaling pathway of melanin biosynthesis induced by α-melanocyte-stimulating hormone (α-MSH).

### TECHNICAL FIELD

The present invention relates to a composition for skin whitening comprising a dihydro-5-methylfuran-2(3H)-one derivative having melanogenesis inhibitory activity, or a pharmaceutically acceptable salt thereof. Particularly, the compound of the present invention acts on a signaling pathway of melanin biosynthesis induced by α-melanocyte-stimulating hormone (α-MSH) to prevent or treat melanogenesis and hyperdeposition disease.

### BACKGROUND ART

Melanin is a black pigment found in animals, plants, microorganisms, etc., which is is a substance that is not essential for growth or development, but that enhances viability and competitiveness in the environment. It is known that melanin is a stable pigment among the pigments found in living organisms, and is insoluble in almost all solvents, and that the process of melanogenesis occurs in melanosomes, which are organelles of melanocytes, specially differentiated cells. That is, skin color is determined by the content, distribution, etc. of melanin, and is associated to the number and distribution of melanosomes that are produced in melanocytes and then released to the outside of the cells.

Melanin exists in the skin and has an important function to protect the body from UV rays, etc., but when melanin is excessively produced, it is known as an important factor that promotes pigmentation and skin aging, and that causes hyperpigmentation such as melisma and freckles, etc., and melanoma.

A person's skin color is determined by several factors such as the activity of melanocytes that produce melanin pigment, the distribution of blood vessels, the thickness of the skin, and the presence or absence of pigments inside and outside human body such as carotenoids and bilirubin.

In particular, the most important factor is the black pigment called melanin, produced by the action of several enzymes such as tyrosinase, etc., in melanocytes. Melanogenesis is influenced by genetic factors, hormone secretion, physiological factors related to stress, and environmental factors such as UV radiation.

Melanin pigment is produced in melanin cells and then moves to keratinocytes to protect the skin from harmful environmental stimuli such as UV radiation. Ultraviolet rays from the sun can be classified according to wavelength into UVA in the 320-400 nm region; UVB in the region of 280-320 nm; and UVC in the 200-280 nm region, of which UVB induces melanogenesis.

UVB induces melanogenesis by complex process through the interaction between keratinocytes and melanin cells, or increases melanogenesis by method increasing the expression of Microphthalmia-associated transcription factor (MITF), which is a major regulator of melanogenesis, through phosphorylation of Ser133 of cAMP-response element-binding protein (CREB) by cAMP increase.

When exposed to UVB, keratinocytes secrete paracrine factor to promote melanogenesis in melanin cells, and among the paracrine factors secreted, the most important agonist that stimulates melanogenesis is α-melanocyte stimulating hormone (α-MSH).

Existing skin whitening agents show whitening efficacy through mechanism to inhibit excessive melanogenesis, and mainly Arbutin, Kojic acid, etc., which inhibit tyrosinase activity, have been used.

However, these whitening agents do not show a great effect when the melanogenesis is increased through the secretion of neurotransmitters due to internal factors such as mental stress, lack of sleep or fatigue, and these inhibitors have a problem of showing weak activity, or side effects which degenerate pigment cells to impair the original function of cells. In addition, it is well known that side effects such as vitiligo may occur when the activity of tyrosinase is inhibited. (1. A comprehensive review on tyrosinase inhibitors, Journal of Enzyme Inhibition and Medicinal Chemistry, VOL. 34, NO. 1, 2019; 2. Potential adverse effect of tyrosinase inhibitors on teleosts:A review, Comparative Biochemistry and Physiology, Part C, VOL. 228, 2020).

Therefore, there is an urgent need to develop a new inhibitor capable of inhibiting melanogenesis with low cytotoxicity and without side effects due to inhibition of tyrosinase activity.

On the other hand, maliolide is a natural product first isolated from the hexane extract of the leaves of Mollinedia marliae. Korean Patent No. 1890058 discloses a composition for preventing, inhibiting or treating chronic inflammatory diseases, comprising maliolide derivatives. However, the literature never discloses whether maliolide and its derivatives are involved in melanogenesis inhibition or melanosome degradation.

Accordingly, the present inventors confirm that during trying to develop a substance having melanosome degradation promoting efficacy superior to the above-mentioned conventional melanogenesis inhibitor, dihydro-5-methylfuran-2(3H)-one derivatives exhibit excellent effects in promoting degradation of melanosome induced by α-melanocyte stimulating hormone (α-MSH) without cytotoxicity and side effects.

### DETAILED DISCLOSURE

### Technical Problem

The present inventors studied to develop a substance having the effect of inhibiting melanogenesis and promoting melanosome degradation, and as a result, completed the present invention by confirming that dihydro-5-methylfuran-2(3H)-one derivatives had an excellent effect.

An object of the present invention is to provide a composition for skin whitening comprising a dihydro-5-methylfuran-2(3H)-one derivative or a pharmaceutically acceptable salt thereof as an active ingredient, which have no cytotoxicity and a low risk of side effects due to inhibition of tyrosinase activity, and inhibit melanogenesis and promote melanosome degradation.

In addition, another object of the present invention is to provide a composition for skin whitening comprising a dihydro-5-methylfuran-2(3H)-one derivative and hydroquinone, which inhibit melanogenesis and promote melanosome degradation.

### Technical Solution

The present invention provides a composition comprising a dihydro-5-methylfuran-2(3H)-one derivative of the following Formula (I), an isomer thereof, or a pharmaceutically acceptable salt thereof.

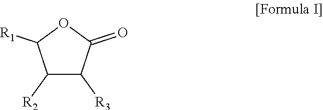

wherein

R₁ is hydrogen, C₁-C₆ alkyl, C₂-C₆ alkenyl, or C₂-C₆ alkynyl;

R₂ is hydrogen, C₁-C₆ alkyl, C₂-C₆ alkenyl, C₂-C₆ alkynyl, halo, hydroxy, C₁-C₆ alkoxy, cyano, or —ORₐ;

R₃ is C₂-C₂₀ alkyl, which forms single bond or double bond with the carbon of the adjacent furan ring;

Rₐ is —C(O)R_{b}, or Si unsubstituted or substituted with C₁-C₆ alkyl;

R_{b} is hydrogen or C₁-C₆ alkyl.

The composition of the present invention may be a cosmetic composition for skin whitening, a health functional food for skin whitening, or a pharmaceutical composition for the treatment or prevention of melanin pigment hyperdeposition disease.

### Advantageous Effects

The dihydro-5-methylfuran-2(3H)-one derivatives of the present invention have no cytotoxicity and inhibit melanogenesis, and thereby can be usefully used for skin whitening, or improvement, prevention or treatment of melanin pigment hyperdeposition diseases.

In addition, the dihydro-5-methylfuran-2(3H)-one derivatives of the present invention have an effect of reducing melanin by mechanism of action that does not inhibit the activity of tyrosinase, and thus have an excellent effect with a lower risk of side effects compared to existing skin whitening agents that inhibit the activity of tyrosinase to be able to cause side effects such as vitiligo.

### BRIEF DESCRIPTION OF DRAWINGS

FIGS. 1 and 2 show the viability of B16 melanoma cells according to the treatment concentration of dihydro-5-methylfuran-2(3H)-one derivatives.

FIGS. 3 and 4 show the melanogenesis inhibitory effect of dihydro-5-methylfuran-2(3H)-one derivatives.

FIG. 5 shows the inhibitory effect on melanogenesis when hydroquinone and dihydro-5-methylfuran-2(3H)-one derivatives were treated alone or in combination.

FIG. 6 shows the viability of B16 melanoma cells when hydroquinone and dihydro-5-methylfuran-2(3H)-one derivatives were treated alone or in combination.

FIG. 7 shows the result of confirming whether dihydro-5-methylfuran-2(3H)-one derivatives inhibit tyrosinase activity.

### MODE FOR THE INVENTION

Hereinafter, embodiments and examples of the present invention will be described in detail with reference to the accompanying drawings so that those of ordinary skill in the art to which the present invention pertains can easily carry out. However, the present application may be embodied in various forms and is not limited to the embodiments and examples described herein.

Throughout this specification, when a part “comprises” a component, it means that other components may be further included, rather than excluding other components, unless otherwise stated.

The present invention relates to a composition comprising a dihydro-5-methylfuran-2(3H)-one derivative of the following Formula I, an isomer thereof, or a pharmaceutically acceptable salt thereof.

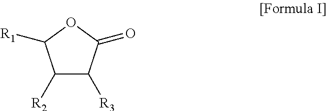

wherein

R₁ is hydrogen, C₁-C₆ alkyl, C₂-C₆ alkenyl, or C₂-C₆ alkynyl;

R₂ is hydrogen, C₁-C₆ alkyl, C₂-C₆ alkenyl, C₂-C₆ alkynyl, halo, hydroxy, C₁-C₆ alkoxy, cyano, or —ORₐ;

R₃ is C₂-C₂₀ alkyl, which forms single bond or double bond with the carbon of the adjacent furan ring;

Rₐ is —C(O)R_{b}, or Si unsubstituted or substituted with C₁-C₆ alkyl;

R_{b} is hydrogen or C₁-C₆ alkyl.

In one embodiment, R₁ is methyl;

R₂ is hydrogen, hydroxy, or —ORₐ;

R₃ is C₁₀-C₁₆ alkyl, which forms double bond with the carbon of the adjacent furan ring;

Rₐ is —C(O)R_{b}, or Si unsubstituted or substituted with C₁-C₆ alkyl;

R_{b} can be C₁-C₆ alkyl.

The composition may be a cosmetic composition for skin whitening, a health functional food for skin whitening, or a pharmaceutical composition for the treatment or prevention of melanin pigment hyperdeposition disease.

In one embodiment, the compound of the present invention is a compound represented by the following Formula II, an isomer thereof, or a pharmaceutically acceptable salt thereof.

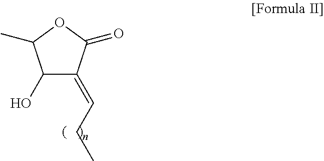

wherein n is an integer from 0 to 18, preferably an integer from 8 to 14, more preferably an integer from 11 to 13.

In one embodiment, the compound of the present invention is a compound represented by the following Formula III, an isomer thereof, or a pharmaceutically acceptable salt thereof.

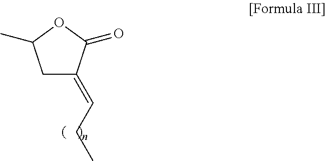

wherein n is an integer from 0 to 18, preferably an integer from 8 to 14, more preferably an integer from 11 to 13.

In one embodiment, the compound of the present invention is a compound selected from the group consisting of the following formulas, an isomer thereof, or a pharmaceutically acceptable salt thereof:

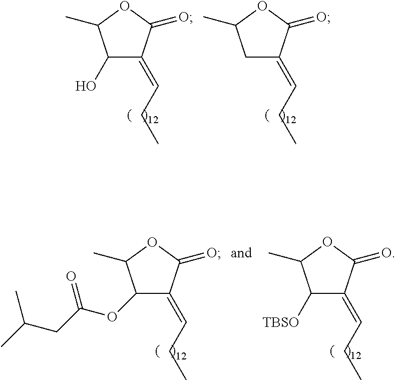

In one embodiment, the compound of the present invention is a compound selected from the group consisting of the following formulas, an isomer thereof, or a pharmaceutically acceptable salt thereof:

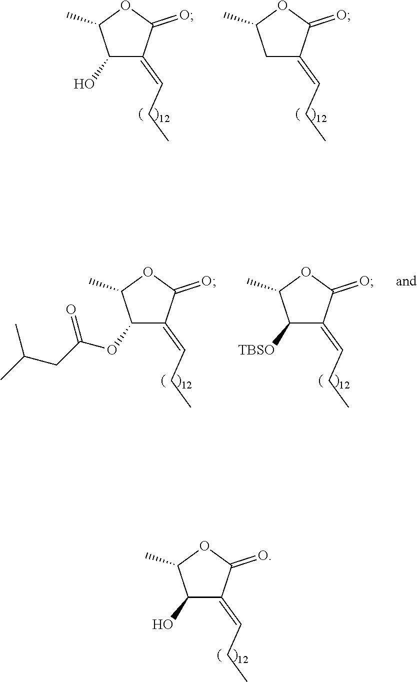

The compound of the present invention inhibits melanogenesis or promotes melanosome degradation, and may be, for example, acting on a signaling pathway of melanin biosynthesis induced by α-MSH.

“Melanin pigment hyperdeposition disease” of the present invention may be melisma, freckles, spots, solar surplus, drug pigmentation, inflammatory pigmentation, senile pigmentation or gestational pigmentation, but is not limited thereto.

A cosmetic composition for skin whitening, a health functional food for skin whitening, or a pharmaceutical composition for the treatment or prevention of melanin pigment hyperdeposition disease of the present invention can further comprise hydroquinone (Benzene-1,4-diol). Hydroquinone is a skin whitening agent that inhibits the conversion of dopa to melanin by suppressing the action of tyrosinase, and thereby inhibits excessive pigmentation of the skin such as melisma and freckles. This exhibits an action to inhibit the enzyme that melanin cells require to produce pigment in the epidermal layer of the skin. As a result, the production of melanin pigment is reduced, and the excessively pigment-deposited skin is discolored.

In addition, the composition of the present invention may further include mulberry extract, Arbutin, ethyl ascorbyl ether, oil-soluble licorice extract, ascorbyl glucoside, magnesium ascorbyl phosphate, niacinamide, alpha-bisabolol or ascorbyl tetraisopalmitate, etc., which have whitening effect.

As used herein, “alkyl” refers to a hydrocarbon having a primary, secondary, tertiary or quaternary carbon atom, and includes a saturated aliphatic group, which may be straight-chain, branched, or cyclic, or a combination thereof. For example, an alkyl group can have 1 to 20 carbon atoms (i.e., C₁-C₂₀ alkyl), 1 to 10 carbon atoms (i.e., C₁-C₁₀ alkyl), or 1 to 6 carbon atoms (i.e., C₁-C₆ alkyl). Unless defined otherwise, in a preferred embodiment, alkyl refers to C₁-C₆ alkyl. Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-buthyl (n-Bu, n-buthyl, —CH₂CH₂CH₂CH₃), 2-methyl-1-propyl (i-Bu, i-buthyl, —CH₂CH(CH₃)₂), 2-buthyl (s-Bu, s-buthyl, —CH(CH₃)CH₂CH₃), 2-methyl-2-propyl (t-Bu, t-buthyl, —C(CH₃)₃), 1-penthyl (n-penthyl, —CH₂CH₂CH₂CH₂CH₃), 2-penthyl (—CH(CH₃)CH₂CH₂CH₃), 3-penthyl (—CH(CH₂CH₃)₂), 2-methyl-2-buthyl (—C(CH₃)₂CH₂CH₃), 3-methyl-2-buthyl (—CH(CH₃)CH(CH₃)₂), 3-methyl-1-buthyl (—CH₂CH₂CH(CH₃)₂), 2-methyl-1-buthyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-penthyl (—C(CH₃)₂CH₂CH₂CH₃), 3-methyl-2-penthyl (—CH(CH₃)CH(CH₃)CH₂CH₃), 4-methyl-2-penthyl (—CH(CH₃)CH₂CH(CH₃)₂), 3-methyl-3-penthyl (—C(CH₃)(CH₂CH₃)₂), 2-methyl-3-penthyl (—CH(CH₂CH₃)CH(CH₃)₂), 2,3-dimethyl-2-buthyl (—C(CH₃)₂CH(CH₃)₂), 3,3-dimethyl-2-buthyl (—CH(CH₃)C(CH₃)₃), and octyl (—(CH₂)₇CH₃).

Moreover, the term “alkyl” as used throughout the specification, working examples and claims is intended to include both unsubstituted and substituted alkyl groups, the latter of which refers to an alkyl moiety having a substituent replacing hydrogen on one or more carbons of the hydrocarbon backbone, including haloalkyl groups such as trifluoromethyl and 2,2,2-trifluoroethyl.

The term “C_{x-y}” or “Cₓ-C_{y}” when used in conjunction with a chemical moiety such as alkyl, alkenyl, alkynyl or alkoxy is intended to include groups containing from x to y carbons in the chain. C₀alkyl denotes hydrogen when the group is at terminal position, or a bond when the group is at internal position. For example, C₁-C₆ alkyl group contains 1 to 6 carbon atoms in the chain.

“Alkoxy” refers to a group having the formula —O-alkyl, wherein the alkyl group as defined above is attached to the parent compound through an oxygen atom. The alkyl moiety of the alkoxy group may contain, for example, 1 to 20 carbon atoms (i.e., C₁-C₂₀ alkoxy), 1 to 12 carbon atoms (i.e., C₁-C₁₂ alkoxy), 1 to 10 carbon atoms (i.e., C₁-C₁₀ alkoxy), or 1 to 6 carbon atoms (i.e., C₁-C₆ alkoxy). Examples of suitable alkoxy groups include, but are not limited to, methoxy (—O—CH₃ or —OMe), ethoxy (—OCH₂CH₃ or —OEt), and t-butoxy (—OC(CH₃)₃ or —O-tBu).

“Alkenyl” refers to a hydrocarbon having primary, secondary, tertiary or quaternary carbon atoms, including straight-chain, branched and cyclic groups, or combinations thereof, and having at least one region of unsaturation, i.e., carbon-carbon sp² double bonds. For example, an alkenyl group can have 2 to 20 carbon atoms (i.e., C₂-C₂₀ alkenyl), 2 to 12 carbon atoms (i.e., C₂-C₁₂ alkenyl), 2 to 10 carbon atoms (i.e., C₂-C₁₀ alkenyl), or 2 to 6 carbon atoms (i.e., C₂-C₆ alkenyl). Examples of suitable alkenyl groups include, but are not limited to, vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” refers to a hydrocarbon having primary, secondary, tertiary or quaternary carbon atoms, including straight-chain, branched and cyclic groups, or combinations thereof, and having at least one of carbon-carbon sp triple bonds. For example, an alkenyl group may have 2 to 20 carbon atoms (i.e., C₂-C₂₀ alkynyl), 2 to 12 carbon atoms (i.e., C₂-C₁₂ alkynyl), 2 to 10 carbon atoms (i.e., C₂-C₁₀ alkynyl), or 2 to 6 carbon atoms (i.e., C₂-C₆ alkynyl). Examples of suitable alkynyl groups include, but are not limited to, acetylenic (—C≡CH) and propargyl (—CH₂C≡CH).

As used herein, the term “halo” includes chloro, fluoro, bromo, and iodo.

As used herein, “isomer” is meant to include diastereomers and enantiomers.

In the present specification, “TBS” means tert-butyldimethylsilyl.

As used herein, a “pharmaceutically acceptable salt” refers to a salt according to an aspect of the present invention that is pharmaceutically acceptable and has the desired pharmacological activity of the parent compound. In addition, pharmaceutically acceptable salts herein are intended to refer to all salts that can be used in pharmaceutical compositions as well as cosmetic compositions or food compositions. The salt is not particularly limited as long as it is pharmaceutically acceptable, and for example, hydrochloric acid, sulfuric acid, nitric acid, phosphoric acid, hydrofluoric acid, hydrobromic acid, formic acid, acetic acid, tartaric acid, lactic acid, citric acid, fumaric acid, maleic acid, succinic acid, methanesulfonic acid, benzenesulfonic acid, toluenesulfonic acid, naphthalenesulfonic acid, etc. can be used.

A cosmetic composition of the present invention, for example, may be prepared in the form of solution, gel, solid or kneaded anhydrous product, emulsion, suspension, microemulsion, microcapsule, microgranule, or ionic (liposome), non-ionic type of vesicular dispersant, obtained by dispersing an oil phase in an aqueous phase, in the form of cream, skin, lotion, powder, ointment, spray or concealer stick. In addition, it may be prepared in the form of a foam or in the form of an aerosol composition further containing a compressed propellant.

In addition, the cosmetic composition may contain fatty substances, organic solvents, solubilizers, thickening agents and gelling agents, emollients, antioxidants, suspending agents, stabilizers, foaming agents, fragrances, surfactants, water, an ionic or non-ionic type emulsifiers, fillers, sequestering and chelating agents, preservatives, vitamins, blocking agents, wetting agents, essential oils, dyes, pigments, hydrophilic or lipophilic actives, lipid vesicles, or adjuvants commonly used in the cosmetics field such as any other ingredients commonly used in cosmetics.

In another embodiment, the composition of the present invention may be a food composition, for example, a health functional food. The “health functional food” means a food manufactured and processed using raw materials or ingredients with functionality useful for the human body, and the “functionality” means to have intake for the purpose of regulating nutrients with respect to the structure and function of the human body or obtaining useful effects a health purpose such as physiological action.

The formulation of the food composition is not particularly limited, but, for example, may be formulated as tablets, granules, powders, liquids such as drinks, caramel, gels, bars, and the like. In addition to the active ingredient, the food composition of each formulation can be formulated by appropriately selecting ingredients commonly used in the art without difficulty by those skilled in the art according to the formulation or purpose of use.

The pharmaceutical composition of the present invention may be administered parenterally or orally according to a desired method, and the dosage may vary depending on the patient's weight, age, sex, health condition, diet, administration time, administration method, excretion rate, severity of disease, etc. In addition, the therapeutically effective amount of the composition may vary depending on the method of administration, the target site, and the condition of the patient, and when used in the human body, the dosage should be determined as an appropriate amount in consideration of both safety and efficiency.

The present invention will be described in more detail through the following working examples, but the following working examples are for illustrative purposes only and are not intended to limit the scope of the present invention.

In the following working examples, compounds 1 to 5 refer to the following compounds, respectively.

TABLE 1

| No. | Structure |
|---|---|
| Compound 1 | 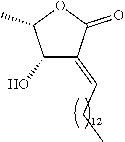 |
| Compound 2 | 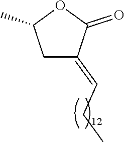 |
| Compound 3 | 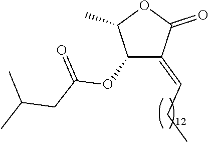 |
| Compound 4 | 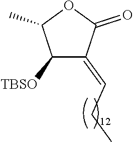 |
| Compound 5 | 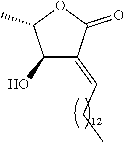 |

### REFERENCE EXAMPLE 1

Cell Culture

After diluting B16 melanoma cells with 0.25% trypsin solution, the cells were isolated. The isolated cells were put in Dulbecco's modified eagle's medium (DMEM) supplemented with 10% fetal bovine serum (FBS) and 1% penicillin/streptomycin (100 U/ml), adapted to 37° C., 5% CO₂ incubator and cultured.

### EXAMPLE 1

Analysis of Effects on Cell Viability

Using B16 melanoma cells, cytotoxicity was measured by MTT assay as follows.

200 μL of B16 melanoma cells were aliquoted in a 96-well plate to 2.7×10³ cells/well, and then cultured at 37° C., 5% CO₂ incubator for 24 hours. B16 melanoma cells were treated with Compounds 1 to 5 at concentrations of 0.3, 1, 3, and 10 μM for 2 hours.

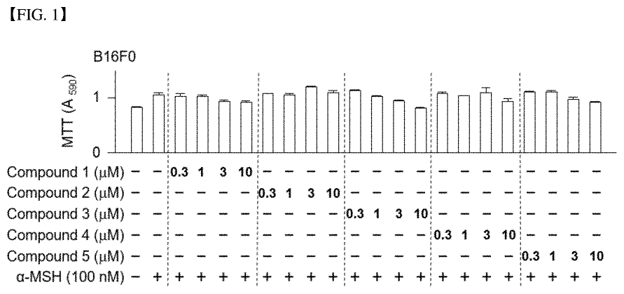

Thereafter, α-MSH was treated at a concentration of 100 nM for 76 hours, and absorbance was measured at 590 nm by MTT assay, and the results are shown in FIG. 1. In addition, absorbance was measured in the same manner for 1, 3, and 10 μM of racemic mixtures of Compound 1, and the results are shown in FIG. 2.

As shown in FIGS. 1 and 2, all of the Compounds of the present invention exhibited the equivalent level of cell viability as compared to the control group, and it is confirmed that there was no cytotoxicity.

### EXAMPLE 2

Melanogenesis Inhibition and Melanosome Degradation Effect

Using B16 melanoma cells, melanogenesis inhibition and melanosome degradation effect were confirmed as follows.

B16 melanoma cells were aliquoted in a 96-well culture plate at 2.7×10³ cells/well. After 24 hours, medium from each 96-well was removed and treated with DMEM medium containing 10% FBS and with Compounds 1 to 5 at concentrations of 0.3, 1, 3, and 10 μM for 2 hours. Thereafter, α-MSH was treated at a concentration of 100 nM for 72 hours.

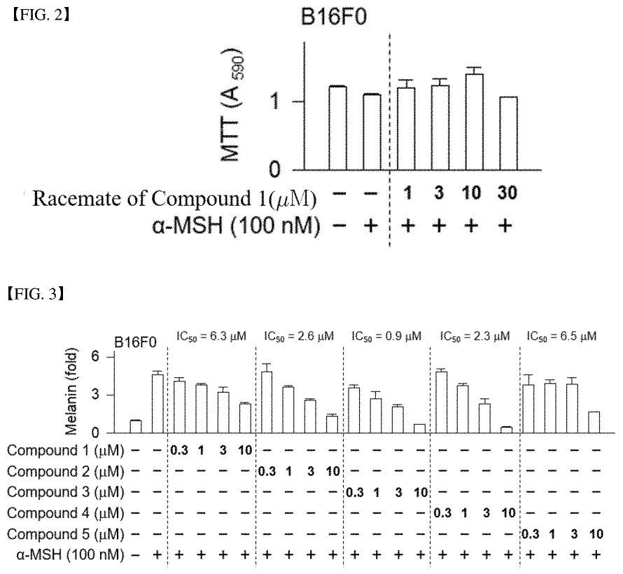

After 72 hours, the absorbance was measured at 405 nm to measure the amount of melanin released into the medium and the results are shown in FIG. 3. In addition, absorbance was measured in the same manner for 1, 3, 10, and 30 μM of racemic mixtures of Compound 1, and the results are shown in FIG. 4.

As shown in FIGS. 3 and 4, as compared to the positive control treated with α-MSH, when the compound of the present invention was treated, melanogenesis inhibition and melanosome degradation effect were concentration-dependently increased, and thereby it was confirmed to have excellent melanogenesis inhibition and melanosome degradation effect.

### EXAMPLE 2

Melanogenesis Inhibition and Melanosome Degradation Effect

Using B16 melanoma cells, melanogenesis inhibition and melanosome degradation effect of the compounds of Table 2 below were confirmed.

B16 melanoma cells were aliquoted in a 96-well culture plate at 2.7×10³ cells/well. After 24 hours, medium from each 96-well was removed and treated with DMEM medium containing 10% FBS, serially diluted samples, and stimulant (α-MSH), respectively. After 72 hours, absorbance was measured at 405 nm to quantify the amount of melanin released into the medium. The inhibition rate was calculated by subtracting the amount of melanin in the cells treated only with the medium from the amount of melanin in the cells treated with the stimulant as a control. The standard curve used for quantification of melanin was prepared by dissolving melanin synthesized in 0.85M KOH solution, serially diluting it with DMEM 10% FBS medium, aliquoting it in a 96-well culture plate, and then measuring the absorbance at 405 nm. Using this value, the amount of melanin released out of the cell was directly calculated.

As compared to the positive control treated with a-MSH alone, when the compounds of the present invention were treated, melanogenesis inhibition and melanosome degradation effect are shown in Table 2 below.

TABLE 2

| 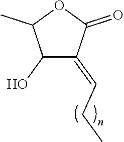 | Melanin Inhibition rate (%) | 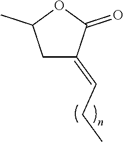 | Melanin Inhibition rate (%) |
|---|---|---|---|
| n = 8 | 5.2 | n = 8 | 10.3 |
| n = 9 | 6.3 | n = 9 | 5.6 |
| n = 10 | 15.6 | n = 10 | 8.5 |
| n = 11 | 30.2 | n = 11 | 26.2 |
| n = 12 | 35.8 | n = 12 | 28.0 |
| n = 13 | 25.3 | n = 13 | 30.2 |
| n = 14 | 11.2 | n = 14 | 4.5 |

As shown in Table 2, it was confirmed that all of the compounds have melanogenesis inhibition effect. In addition, it can be seen that, among the compounds, the compounds wherein n of 11 to 13 exhibite an inhibition rate of 25% or more compared to the positive control group, and have particularly excellent melanogenesis inhibition and melanosome degradation effect.

### EXAMPLE 3

Melanogenesis Inhibition and Melanosome Degradation Effect Upon Combination Treatment with Hydroquinone

Using B16 melanoma cells, melanogenesis inhibition and melanosome degradation effect were confirmed upon single or combination treatment with hydroquinone and the compound of the present invention.

B16 melanoma cells were treated alone or in combination with hydroquinone and/or a compound of the present invention for 2 hours under the following conditions.

(i) Compound 2: 1, 3, 10 μM single administration

(ii) Hydroquinone: 1, 3, 10 μM single administration

(iii) Compound 2 (1, 3, 10 μM)+Hydroquinone (5 μM)

Thereafter, α-MSH was treated at a concentration of 100 nM for 76 hours.

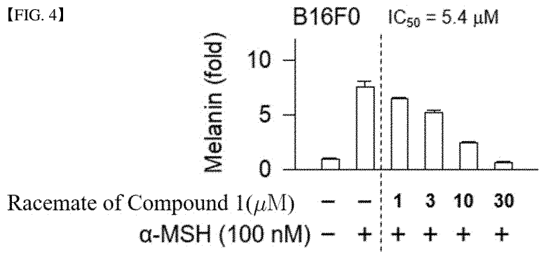

After 72 hours, absorbance was measured at 405 nm to quantify the amount of melanin released into the medium, and the inhibition rate was calculated and shown in FIG. 5.

As shown in FIG. 5, in the case of the positive control treated with α-MSH, the amount of melanin increased 16.5 times, but when Compound 2 and hydroquinone were treated alone or in combination, both melanogenesis inhibition and melanosome degradation effect were dose-dependently increased.

In addition, based on the amount of melanogenesis in the positive control group, the hydroquinone-treated experimental group had an IC₅₀ value of 7.7 μM, and the experimental group treated with Compound 2 had an IC₅₀ value of 3.7 μM. In particular, the IC₅₀ value of the experimental group treated with 5 μM of hydroquinone and Compound 2 in combination was measured to be 1.5 μM, so that it can be seen that melanogenesis inhibition and melanosome degradation effect are excellent more than twice compared to that of the case of administering Compound 2 alone.

### EXAMPLE 4

Cytotoxicity Analysis of Hydroquinone and Compound 2

Using B16 melanoma cells, cytotoxicity was measured by MTT assay when hydroquinone and Compound 2 were treated alone or in combination.

200 μL of B16 melanoma cells were aliquoted in a 96-well plate to 2.7×10³ cells/well, and then cultured at 37° C., 5% CO₂ incubator for 24 hours. B16 melanoma cells were treated with hydroquinone and/or Compound 2 for 2 hours under the same conditions as in Example 3.

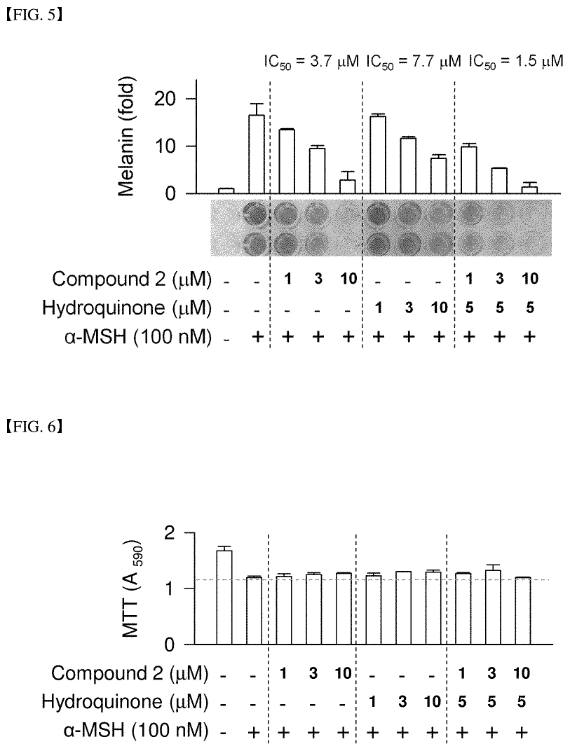

Thereafter, α-MSH was treated at a concentration of 100 nM for 76 hours, and absorbance was measured at 590 nm by MTT assay, and the results are shown in FIG. 6.

As shown in FIG. 6, all of the experimental group treated with hydroquinone alone, the experimental group treated with Compound 2 alone, and the experimental group treated with said substances in combination showed at a concentration of 10 μM or less the equivalent level of cell viability compared to the control group, so that it was confirmed that there is no cytotoxicity.

### Example 5

Confirmation of Inhibition of Tyrosinase Activity

As skin whitening agents, substances that inhibit the activity of tyrosinase are used, but since it is well known that side effects such as vitiligo may occur when the activity of tyrosinase is inhibited, and thus it was confirmed whether Compound 2 of the present invention inhibits the activity of tyrosinase by Enzyme activity measurement as follows.

B16 melanoma cells were aliquoted in a 100 mm culture dish at 2×10⁵ cells/dish. After 24 hours, the medium from the 100 mm culture dish was removed and treated with DMEM medium containing 10% FBS and a stimulant (α-MSH). After 48 hours, tyrosinase was extracted from B16 melanoma cells and used as an enzyme source. Using the principle that tyrosinase oxidizes L-DOPA to DOPA chrome and DOPA chrome has absorbance at 475 nm, tyrosinase and L-DOPA were incubated at 37° C. for 1 hour and absorbance was measured at 475 nm at 2 minute intervals, so that the production amount of DOPA chrome according to time was measured. The amount of changing 1 nmole of L-DOPA per minute to DOPA chrome was defined as the amount of tyrosinase activity. The tyrosinase inhibitory effect of Compound 2 was evaluated by comparing the group treated with tyrosinase and L-DOPA in combination as a positive control and the group treated with tyrosinase, L-DOPA and Compound 2 in combination.

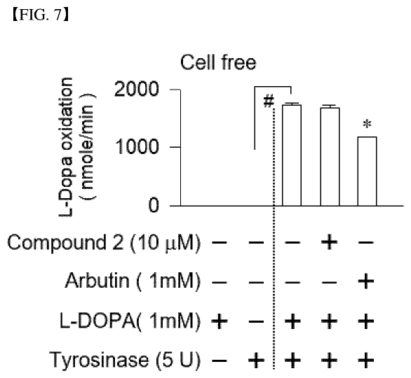

As shown in FIG. 7, unlike the existing skin whitening agents, Arbutin, which inhibited the activity of tyrosinase, it was confirmed that Compound 2 showed the equivalent level of tyrosinase activity to that of the positive control treated with tyrosinase and L-DOPA, and thus did not inhibit the activity of tyrosinase.

After all, the compound of the present invention has the effect of reducing melanin without inhibiting the activity of tyrosinase, so that it can be seen that it has an excellent effect with a lower risk of side effects compared to existing skin whitening agents.

1. A composition for skin whitening comprising a compound represented by the following Formula I, an isomer thereof, or a pharmaceutically acceptable salt thereof: 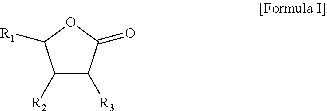
wherein
R₁ is hydrogen, C₁-C₆ alkyl, C₂-C₆ alkenyl, or C₂-C₆ alkynyl;
R₂ is hydrogen, C₁-C₆ alkyl, C₂-C₆ alkenyl, C₂-C₆ alkynyl, halo, hydroxy, C₁-C₆ alkoxy, cyano, or —ORₐ;
R₃ is C₂-C₂₀ alkyl, which forms single bond or double bond with the carbon of the adjacent furan ring;
Rₐ is —C(O)R_{b}, or Si unsubstituted or substituted with one or more C₁-C₆ alkyl; and
R_{b} is hydrogen or C₁-C₆ alkyl.

2. The composition of claim 1, wherein
R₁ is methyl;
R₂ is hydrogen, hydroxy, or —ORₐ;
R₃ is C₁₀-C₁₆ alkyl, which forms double bond with the carbon of the adjacent furan ring;
Rₐ is —C(O)R_{b}, or Si unsubstituted or substituted with one or more C₁-C₆ alkyl; and
R_{b} is C₁-C₆ alkyl.

3. The composition of claim 1, wherein the compound is represented by the following Formula II, an isomer thereof, or a pharmaceutically acceptable salt thereof: 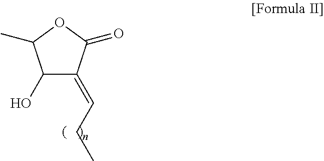
wherein n is an integer from 0 to 18.

4. The composition of claim 3, wherein n is an integer from 8 to 14.

5. The composition of claim 1, wherein the compound is represented by the following Formula III, an isomer thereof, or a pharmaceutically acceptable salt thereof: 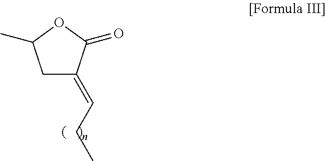
wherein n is an integer from 0 to 18.

6. The composition of claim 5, wherein n is an integer from 8 to 14.

7. The composition of claim 1, wherein the compound is a compound selected from the group consisting of the following compounds, an isomer thereof, or a pharmaceutically acceptable salt thereof:

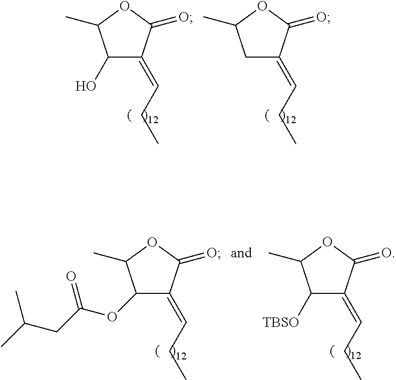

8. The composition of claim 1, wherein the compound inhibits melanogenesis or promotes melanosome degradation.

9. A health functional food for skin whitening comprising the composition of claim 1.

10. A pharmaceutical composition for the treatment or prevention of melanin pigment hyperdeposition disease comprising the composition of claim 1.

11. (canceled)

12. The pharmaceutical composition of claim 10, wherein the melanin pigment hyperdeposition disease is selected from the group consisting of melisma, freckles, spots, solar surplus, drug pigmentation, inflammatory pigmentation, senile pigmentation and gestational pigmentation.

13. The composition of claim 1, further comprising hydroquinone.

14. The health functional food of claim 9, further comprising hydroquinone.

15. The pharmaceutical composition of claim 10, further comprising hydroquinone.

16. A cosmetic composition for skin whitening comprising the composition of claim 1.

17. The cosmetic composition of claim 16, further comprising hydroquinone.

18. A method for whitening skin, the method comprising administering the composition of claim 1.

19. A method of treating melanin pigment hyperdeposition disease, the method comprising administering the pharmaceutical composition of claim 10 to a subject in need thereof.

20. The method of claim 19, wherein the melanin pigment hyperdeposition disease is selected from the group consisting of melisma, freckles, spots, solar surplus, drug pigmentation, inflammatory pigmentation, senile pigmentation and gestational pigmentation.
